(19) United States
(12) Patent Application Publication
(10) Pub. No.: US 2019/0119705 A1
(43) Pub. Date: Apr. 25, 2019

(54) SUBSTANTIALLY NON-RACEMIC KETONE ESTER OF BETA HYDROXYBUTYRATE AND PRODUCTION PROCESS THEREOF

(71) Applicant: KetoneAid Inc., Falls Church, VA (US)

(72) Inventors: Frank Borges LLosa, Falls Church, VA (US); Stephen Zarpas, Washington, DC (US)

(73) Assignee: KetoneAid Inc., Falls Church, VA (US)

(21) Appl. No.: 16/167,449

(22) Filed: Oct. 22, 2018

Related U.S. Application Data
(60) Provisional application No. 62/575,023, filed on Oct. 20, 2017.

(51) Int. Cl. C12P 7/62; A23L 29/00; C07C 67/54

(52) U.S. Cl. CPC C12P 7/62; A23L 29/035; C07C 67/54; C12Y 301/01; A23V 2200/316; A23V 2200/322; A23V 2200/328; A23V 2002/00

(57) ABSTRACT

The embodiments presented herein provide for a method for the production of a substantially non-racemic ketone ester of beta hydroxybutyrate comprising the steps of reacting one part hydroxybutyrate ester with two parts racemic 1,3-butanediol in the presence of a stereoselective lipase.

### CROSS-REFERENCE TO RELATED APPLICATIONS

This application claims priority to U.S. Provisional Application 62/575,023 filed Oct. 20, 2017 which is hereby incorporated by reference.

### TECHNICAL FIELD

This invention generally relates to a method of production for substantially non racemic and non racemic ketone ester of beta hydroxy butyrate.

### BACKGROUND

Ketosis is a fat-based metabolism, a state indicated by elevated levels of ketones in the blood and in which a person's body produces ketones for fueling metabolism rather than primarily using glycogen to make glucose. The ketogenic diet, which can initiate and maintain ketosis, was developed initially to treat pediatric refractory epilepsy. The original diet required ingesting calories primarily from fat, with a minimally sufficient amount of proteins to allow for growth and repair, and with a very restricted amount of carbohydrates. A typical diet would include a 4:1 ratio of fat to combined protein and carbohydrate (by weight). The ketogenic diet can allow one's body to consume fats for fuel rather than carbohydrates. Normally, the carbohydrates contained in food are stored as glycogen in the body and then, when needed, converted into glucose. Glucose is particularly important in fueling brain-function.

When a body lacks carbohydrates, the liver converts fat into fatty acids and ketone bodies. The ketone bodies pass into the brain and replace glucose as an energy source. An elevated level of ketone bodies in the blood, i.e. ketosis, has been shown to reduce the frequency of epileptic seizures. Ketosis may also improve brain-function when a person's body cannot properly use glucose, such as in Alzheimer's patients and those with concussions or other brain damage.

In addition to improved brain-function, ketones can improve muscle performance, such as in endurance athletes. This is because the body can only store and convert about 100-minutes' worth of glycogen into useful glucose during extreme and prolonged exercise, such as in bicycle races and long-distance running. Athletes can train to extend their body's capacity, but there are limits. Yet, with a second or alternative source of energy, from ketones, the body can continue to perform beyond the individual's capacity to produce glucose. Further, studies have shown that ketones can improve endurance performance by as much as four percent.

Supplementations with exogenous ketones has become increasingly popular in recent years as benefits therefrom continue to be understood and discovered. Specifically, (R)3-hydroxybutyl-(R)-3-hydroxybutyrate is a very valuable diastereomer medicinal food due to the stereochemistry of the bioactive (R) isomer. A variety of means for the production of these ketone supplements exist. The problem with the process available today in the art for making non racemic d beta hydroxybutyrate, D 1,3-butanediol is the final cost and difficulty of scaling up to commercial volumes. The current manufacturing process requires the very difficult to compound, and very costly (R)-1,3-butanediol as half of the diastereomer.

Multiple teams have spent decades trying to bring down the cost of ketone ester from the current $1,000 per gram pricing available today. None were able to discover this process, and thus it is non obvious.

Lowering the cost of production promotes accessibility to the a more bioactive ketone foodstuff. The following manufacturing process cuts the cost to manufacture significantly, this novel process is described herein.

### SUMMARY OF THE INVENTION

An embodiment of the present invention provides a method for the production of a substantially non-racemic ketone ester of beta hydroxybutyrate. The method includes reacting one part hydroxybutyrate ester with two parts racemic 1,3-butanediol in the presence of a stereoselective lipase.

In one aspect, reacting the one part hydroxybutyrate ester with two parts racemic 1,3-butanediol in the presence of a stereoselective lipase is performed using transesterification.

In one aspect, the stereoselective lipase is novozyme 435.

In another aspect, a method for increasing the enantiomeric excess of a ketone ester by increasing the percentage of secondary ketone ester. The method includes seperating a primary ketone ester and a secondary ketone ester followed by isolating at least a portion of the secondary ketone ester.

In one aspect, the isolation is performed via a distillation. In another aspect, the isolation is performed via a reaction.

In yet another aspect, 1) beta hydroxybutyrate, DL 1,3-butanediol ester is supplies as a foodstuff. The foodstuff may be supplied with one or more flavoring agents. Further, a pharmaceutically or dietetically acceptable carrier can be provided. The foodstuff may also include D beta hydroxybutyrate, D 1,3-butanediol ester.

### DETAILED DESCRIPTION

A detailed explanation of the composition of matter according to preferred embodiments of the present invention are described below. In general, the invention relates to a novel production process for a substantially non racemic and/or non racemic ketone ester of beta hydroxybutyrate.

In an embodiment, the process is comprised of the step of combining roughly 1 part hydroxybutyrate ester with 2 parts racemic 1,3-butanediol in the presence of a stereoselective lipase. The hydroxybutyrate ester may be defined as a non racemic ethyl hydroxybutyrate or methyl hydroxybutyrate.

The highly stereoselective nature of certain lipase enzymes including Novozyme 435 ensures a product with a very high enantiomeric excess of the (D) isomer of 1,3-Butanediol,

Transesterification is commonly used in organic chemistry for the exchange of an organic R′ group of an ester with an organic R′ group of an alcohol. The desired ester exists in both a primary and secondary isomer. The current method of producing the desired ester is by transesterifying D-1,3-butanediol with D ethyl beta hydroxybutyrate.

The desired ester can be synthesized into a completely racemic diastereomer using racemic DL-beta-hydroxybutyrate and DL-1,3-butanediol. The result is an enantiomeric excess of zero.

The process can be further improved by synthesizing a partially racemic enantiomer of the diastereomer of the desired ester using DL-1,3-butanediol and D-beta-hydroxybutyrate using non-stereoselective means with a non-stereoselective lipase or standard chemical esterification reaction. The resulting diastereomer would be D for the beta-hydroxybutyrate and DL for the 1,3-butanediol portion of the molecule forming a partially non-racemic ketone ester. The result is what we consider to be an enantiomeric excess of 40-60%.

The process can be further improved by using a a stereoselective lipase such as Novozyme 435 enzyme which is stereoselective for the D enantiomer of DL-1,3-butanediol, it is possible to form a substantially non-racemic ketone ester by using DL-1,3-butanediol. The lipase enzyme is stereoselective for both the primary and secondary ketone esters. The result is what we consider to be an enantiomeric excess of 51-100%.

The process can be further improved because the Novozyme 435 may be more stereoselective for the secondary ester. Given structural differences of the primary and secondary esters, the product can be more highly purified with respect to the increased enantiomeric purity by isolating the primary ester away from the secondary ester, for example through distillation. The result is what we consider to be an enantiomeric excess of 75-100%.

The D form has been shown to have the highest bioavailability and is considered to be the purest form, resulting in increased benefits to the user. Many of these benefits are well documented in the scientific literature. The L form, while still consumable, and common in many supplements on the market may be hazardous at regularly dosed high concentrations, and may be neutrally beneficial at best.

The resulting novel compound of D beta hydroxybutyrate, DL 1,3-butanediol ester has no CAS # and not naturally found in nature. This may account for as little as 5% and as much as 100% of the final manufactured process.

D beta hydroxybutyrate, DL 1,3-butanediol ester may provide a low cost solution while providing an increase in the feeling of satiety post-consumption. D beta hydroxybutyrate, DL 1,3-butanediol ester may be more slowly metabolised by the body compared to the 100% D form.

In some embodiments, enatiomerically pure esters would be blended with partially racemic esters. The partially racemic esters are far less costly and would lower the price of production. Ketosis has been shown to improve brain-function by providing a critical source of fuel to fuel starved cells due to a pathologically compromised inability to completely oxidize glucose. More specifically, the impairment has been shown to be a disregulator of pyruvate dehydrogenase. That pathologic impairment is very likely at the root of many well-known neurodegenerative diseases such as Alzheimer's Disease, Parkinson's Disease and amyotrophic lateral sclerosis (ALS). The pathologic impairment to process glucose is also very likely at the core of concussions and Traumatic Brain Injury (TBI). The same impairment is most likely at work in otherwise healthy adults who over time begin to exhibit problems with memory and other cognitive loss.

In addition to improved brain-function, ketones can improve muscle performance, such as in endurance athletes, and muscle recovery that would be beneficial to all athletes, including sprinters.

Aspects of recovery include greater protein synthesis after great physical exertion such as weight lifting, or sports. Aspects of the recovery include more rapid return to heart beat variability as a measure of the recovered post-exercise condition. Another aspect of the more rapid recovery includes heightened potentiating of the MTOR Complex (Mammalian Target of Rapamycin). Another aspect of faster recovery is between sets during exercise wherein a person is able to decrease the necessary rest time between sets. Also increased are the total repetitions possible before fatigue wherein many athletes have been able achieve personal records for both the number of repetitions and maximum weight. Mood elevation effects also produce a greater willingness to engage in strenuous activity that otherwise may recover more encouragement.

Based on studies involving rats' hearts, Alzheimer's patients, and other studies, it may be shown that ketone concentrations in the blood above various threshold minima can provide therapeutic effects for a variety of neurological conditions such as Alzheimer's, Parkinson's, ALS, Multiple Sclerosis, traumatic brain injury, epilepsy, and autism, as well as non-neurological conditions such as diabetes types I & II. For example, D beta hydroxybutyrate, DL 1,3-butanediol ester has been shown to act as a fuel substrate and substitute for glucose in diabetics as well as have hormone-like effects such as lowering of insulin levels.

Present embodiments can also be useful in treating hair loss, vision impairment, amyotrophic lateral sclerosis (commonly referred to as ALS or Lou Gehrig's disease), concussions, heart disease, diabetes, and traumatic brain injury, in addition to enhancing physical performance.

Present embodiments can also be useful in treating heart disease. The heart selectively takes up beta hydroxybutyrate over glucose when both substrates are available for use as a fuel. Myocardial output has been demonstrated to increase by an incredible 50% in healthy resting adults. The present embodiments should be indicated to improve myocardial output in all persons suffering from any myocardial impairment and in particular for persons waiting for a heart transplant to improve and prolong the lifespan while waiting.

A compound of the invention as defined above is also suitably formulated into granules or a powder. In this form it can be readily dispersed in water or other liquid such as tea or a soft drink for human subjects to drink, for instance a beverage or drink as described above. It r ray also be encapsulated, tabletted or formulated with a physiologically acceptable vehicle into unit dosage forms. A unit dosage can comprise a therapeutically effective amount of the extract for a single daily administration, or it can be formulated into smaller quantities to provide for multiple doses in a day. The composition may thus, for instance, be formulated into tablets, capsules, syrups, elixirs, enteral formulations or any other orally administrable form.

Examples of physiologically acceptable carriers include water, oil, emulsions, alcohol or any other suitable material.

In addition to reducing plasma levels of fatty acids, a compound of the invention acts on the appetite centres in the brain. In particular, a compound of the invention increases the levels of various anorexigenic neuropeptides (neuropeptides known to be associated with decreased food intake and decreased appetite) in the appetite centres of the brain and also induces higher levels of malonyl CoA, a metabolite associated with decreased appetite and food intake. The invention therefore further provides a compound of the invention as defined above for use in treating a condition where weight loss or weight gain is implicated. For example, the compound may be used in suppressing appetite, treating obesity, promoting weight loss, maintaining a healthy weight or decreasing the ratio of fat to lean muscle in a subject. The subject in each case may be a healthy subject or a compromised subject. A healthy subject may be, for instance, an individual of healthy weight for whom physical performance and/or physical appearance is important. Examples include members of the military, athletes, bodybuilders and fashion models. A compromised subject may be an individual of non-healthy weight, for instance an individual who is overweight, clinically obese or clinically very obese. A compromised subject may alternatively be an individual of healthy or unhealthy weight who is suffering from a clinical Condition, for instance a condition listed below.

Individuals having the goal of weight loss may benefit from low doses, such as less than 1-5 ml of any combination of compounds discussed herein to be beneficial. A low dose may induce endogenous ketone formation the blood by promoting a signaling pathway, while high doses may shut off this endogenous ketone production signaling pathway.

In addition to reducing plasma levels of fatty acids and acting on the appetite centre in the brain, a compound of the invention increases brain metabolic efficiency, by increasing brain phosphorylation potential and the ΔG′ of ATP hydrolysis. A compound of the invention thereby promotes improved cognitive function and can be used to treat cognitive dysfunction or to reduce the effects of neurodegeneration. A compound of the invention also increases the level of the neuropeptide Brain Derived Neurotrophic Factor (BDNF) in both the paraventricular nucleus (the appetite centre of the brain) and the hippocampus (a part of the brain known to be important for memory). As well as decreasing appetite, BDNF is known to prevent apoptosis and promote neuronal growth in basal ganglia and other areas of interest, thus the increased levels of BDNF produced by the compound of the invention are expected to inhibit neurodegeneration, limit neural tissue death after hypoxia or trauma and promote neural tissue growth.

The compounds of the invention are therefore useful for (a) promoting alertness and improved cognitive function, and (b) inhibiting neurodegeneration. The invention therefore further provides a compound of the invention as defined above for use in promoting alertness or improving cognitive function, or in treating cognitive dysfunction.

In another embodiment, a compound of the invention is used to treat a patient suffering from a condition selected from diabetes, hyperpyrexia, hyperthyroidism, metabolic syndrome X, fever and infection, or a geriatric patient.

A compound of the invention may be administered in combination with one or more additional agents, for instance an agent selected from micronutrients and medicaments. The compound of the invention and the additional agent may be formulated together in a single composition for ingestion. Alternatively the compound of the invention and the additional agent may be formulated separately for separate, simultaneous or sequential administration.

When the additional agent is a medicament it may be, for instance, a standard therapy for a condition from which the subject is suffering. For instance, a compound of the invention may be administered in combination with conventional anti-diabetic agents to a subject suffering from diabetes. Conventional anti-diabetic agents include insulin sensitisers such as the thiazolidinediones, insulin secretagogues such as sulphonylureas, biguanide antihyperglycemic agents such as metformin, and combinations thereof.

When the additional agent is a micronutrient it may be, for instance, a mineral, vitamin or antioxidant. Examples include iron, calcium, magnesium, vitamin A, the B vitamins, vitamin C, vitamin D and vitamin E.

As one skilled in the art will appreciate, embodiments of the present invention may be embodied as, among other things, a composition of matter and a method for making compositions of matter. Other embodiments are within the scope of the following claims.

We claim:

1. A method for the production of a substantially non-racemic ketone ester of beta hydroxybutyrate comprising the steps of:
a. reacting one part hydroxybutyrate ester with two parts racemic 1,3-butanediol in the presence of a stereoselective lipase.

2. The method of claim 1, wherein reacting the one part hydroxybutyrate ester with two parts racemic 1,3-butanediol in the presence of a stereoselective lipase is performed using transesterification.

3. The method of claim 1, wherein the stereoselective lipase is novozyme 435.

4. A method for increasing the enantiomeric excess of a ketone ester by increasing the percentage of secondary ketone ester, the method including the steps of:
a. seperating a primary ketone ester and a secondary ketone ester; and
b. isolating at least a portion of the secondary ketone ester.

5. The method of claim 4, wherein isolation is performed via a distillation.

6. The method of claim 4, wherein isolation is performed via a reaction.

7. A foodstuff comprising:
D beta hydroxybutyrate, DL 1,3-butanediol ester.

8. The foodstuff of claim 7, further comprising one or more flavoring agents.

9. The foodstuff of claim 7, further comprising a pharmaceutically or dietetically acceptable carrier.

10. The foodstuff of claim 7 further comprising D beta hydroxybutyrate, D 1,3-butanediol ester.
